(19) United States
(12) Patent Application Publication
(10) Pub. No.: US 2016/0106695 A1
(43) Pub. Date: Apr. 21, 2016

(54) PETROSELINIC ACID OR A COMBINATION OF ACTIVE INGREDIENTS COMPRISING AT LEAST PETROSELINIC ACID FOR PROMOTING WEIGHT LOSS AND/OR WEIGHT MAINTENANCE

(71) Applicant: NESTEC SA, Vevey (CH)

(72) Inventors: Yann MAHE, Sainte Genevieve des Bois (FR); Carole BRU, Courbevoie (FR); Audrey GUENICHE, Rueil Malmaison (FR)

(21) Appl. No.: 14/889,895

(22) PCT Filed: May 6, 2014
(86) PCT No.: PCT/IB2014/061235

§ 371 Date: Nov. 9, 2015

(30) Foreign Application Priority Data
May 7, 2013 (FR) 1354185

(51) Int. Cl. A61K 31/201; A61K 31/191; A61K 33/30; A61K 36/23; A61K 31/593; A61K 31/355; A61K 31/01; A61K 45/06; A61K 31/185; A61K 9/00

(52) U.S. Cl. CPC A61K 31/201; A61K 31/185; A61K 31/191; A61K 33/30; A61K 9/0053; A61K 31/593; A61K 31/355; A61K 31/01; A61K 45/06; A61K 36/23; A23L 1/293; A23L 1/3002; A23L 1/3008; A23L 1/304; A23L 1/30; A23V 2002/00

(57) ABSTRACT

The present invention relates to the field of weight management and obesity. It concerns petroselinic acid or a combination of active ingredients including at least petroselinic acid and at least one compound chosen from zinc, taurine, one of the salts of same, lycopene and the mixtures thereof, and preferably at least taurine or zinc gluconate and, more preferably still, at least taurine and zinc gluconate, as a drug for oral administration or as a functional food intended to promote weight loss and/or weight maintenance in a human being and/or animal, or indeed intended to fight obesity.

The present invention relates to the field of compositions for accompanying weight management, promoting weight loss and/or weight maintenance in man and/or animals.

In particular, the invention is directed toward proposing the use of petroselinic acid or of combinations comprising at least petroselinic acid, which are useful for accompanying weight management, promoting weight loss and/or weight maintenance in man and/or animals. Petroselinic acid or the combination of active agents under consideration in the context of the present invention are preferably used as medicament or in a functional food.

During weight gain, the development of the adipose mass, or hypertrophy of adipose tissue, can evolve between simple local excess weight (lipodysmorphia) and the formation of cellulite, passing through a certain level of stoutness and ending in actual obesity. Obesity is a real incapacitating pathological condition when it results in particular in the development of a metabolic syndrome.

Thus, in recent decades, the prevalence of obesity has increased worldwide to epidemic proportions. About 1 billion people worldwide are overweight or obese, these conditions increasing mortality, mobility and costs. Obesity develops when the energy intake is greater than the energy expenditure, the excess energy being stored mainly in the form of fat in the adipose tissue. Loss of body weight and prevention of weight gain may be achieved by reducing the energy intake or the bioavailability, by increasing the energy expenditure and/or by reducing the storage in the form of fat. Obesity constitutes a serious threat to health since it is associated with a range of chronic diseases, including diabetes, atherosclerosis, degenerative diseases, respiratory pathway diseases and certain cancers.

In biological terms, an inflammatory state of the adipose tissue may be observed during adipose tissue hypertrophy. Inflammation of the adipose tissue, in particular of the subcutaneous white adipose tissue (ScWAT), has been particularly well described in the case of obesity. Indeed, when the energy balance of the body is unbalanced, either through a lack of physical exercise or through excessive consumption of food (or both), the subcutaneous adipose tissue expands and accumulates under the skin. When this significant development of the adipose mass is maintained, a more general metabolic imbalance may follow. The adipose tissue is in fact considered, as a whole, to be an important endocrine organ, the physiology of which may be impaired by adipose cell hypertrophy and the accumulation of periadipocyte immune cells, including in particular macrophages.

It has thus been described that the pre-adipocytes of non-obese women respond to the factors produced by these macrophages and produce molecules and chemokines such as IL-8 and MCP1 which further amplify and maintain the adipose tissue inflammation by recruiting further inflammatory cells into the adipose tissue (D. Lacasa et al. Endocrinology 148(2): 868-87 (2007); M. Keophiphat et al. Molecular endocrinology 23:11-24 (2009).

As an expected and ubiquitous physiological consequence of such a chronic inflammatory state associated in particular with an overly large development or else with an overly fast variation in size of the adipose tissue (both upward and downward), a “pro-fibrotic” phenotype may then develop in the inflammatory adipose tissue.

As illustrated in the examples of the present application, the inventors have indeed noted that petroselinic acid or a combination of active agents in accordance with the invention proves to be capable of synergistically increasing the amount of lipoxin A4. Lipoxin A4 belongs to the resolvin family. This family of compounds naturally produced by the body acts in a manner complementary to conventional anti-inflammatory agents by raising the threshold for triggering a “dermatologically conventional” inflammatory response, and more particularly so as to raise the threshold of appearance of the signals of this conventional inflammation, namely redness, pain and heat.

Consequently, lipoxin A4 appears to be a potential target for acting on adipose tissue.

Consequently, the present invention is more particularly focused on identifying active agents or combinations of active agents that exert a significant action on lipoxin A4.

The use of petroselinic acid for preparing a composition intended for activating the peroxisomal β-oxidation of fatty acids in the superficial tissues of a mammal, so as to be able to treat or prevent inflammations and/or modulate lipid metabolism in these superficial tissues, is known from document EP 888 773. The skin conditions more particularly targeted in said document are inflammations associated with psoriasis, erythema (sunburn), eczema, seborrhoeic dermatitis, alopecia areata, mycosis, acne or other forms of dermatosis.

Thus, none of the prior art documents suggests that petroselinic acid or a combination of active agents comprising at least petroselinic acid and at least one compound chosen from zinc, taurine, a salt thereof, lycopene, and mixtures thereof, exerts activity on lipoxin A4.

A first subject of the invention is thus petroselinic acid or a combination of active agents comprising at least petroselinic acid and at least one compound chosen from zinc, taurine, a salt thereof, lycopene, and mixtures thereof, preferentially at least taurine or zinc gluconate, and even more preferably at least taurine and zinc gluconate, as a medicament for oral administration or as a functional food for promoting weight loss and/or weight maintenance in man and/or animals.

A subject of the invention is also petroselinic acid or a combination of active agents comprising at least petroselinic acid and at least one compound chosen from zinc, taurine, a salt thereof, lycopene, and mixtures thereof, preferentially at least taurine or zinc gluconate, as a medicament for oral administration or as a functional food for reducing the weight of fat mass in man and/or animals.

According to the present invention, the term “fat mass” is intended to denote the mass of adipose tissue, or fat, in man or animals, as opposed to the muscle mass.

A subject of the invention is also petroselinic acid or a combination of active agents comprising at least petroselinic acid and at least one compound chosen from zinc, taurine, a salt thereof, lycopene, and mixtures thereof, preferentially at least taurine or zinc gluconate, as a medicament for oral administration or as a functional food for reducing the total weight of a human and/or animal.

The invention is also directed toward petroselinic acid or a combination of active agents comprising at least petroselinic acid and at least one compound chosen from zinc, taurine, a salt thereof, lycopene, and mixtures thereof, preferentially at least taurine or zinc gluconate, and even more preferably at least taurine and zinc gluconate, as a medicament for oral administration or as a functional food for combating obesity.

The weight modification, and in particular weight loss, may be due to a slimming diet or to pregnancy.

In the context of the present invention, the following terms have more particularly been defined:

The term “preventing” is intended to mean “reducing the risk of developing”.

The term “functional food” or “food products” means a food that is similar in appearance to a conventional food or a conventional food forming part of the normal diet and for which it has been demonstrated that it affords, beyond the basic nutritional functions, physiological benefits specified by scientific documentation and that it reduces the risk of chronic diseases. Functional food as defined above also includes drinks.

The term “BMI” or “body mass index” means the ratio of the weight in kg divided by the square of the height in meters.

The term “overweight” is used for a human adult with a BMI of between 25 and 30.

The term “obesity” means a condition in which the natural energy reserve, stored in the adipose tissue of animals, in particular man and other mammals, is increased such that it is associated with a certain state of health or increased mortality. An adult human with a BMI of greater than 30 is considered as “obese”.

The term “weight loss” relates to a reduction of the body mass of a human and/or an animal. Weight loss may be in keeping with concern for improving the health, the fitness level and/or the appearance.

The term “weight management” or “weight maintenance” means maintaining the total body mass. For example, the weight management may be relative to maintaining a BMI of between 18.5 and 25, this range being considered as normal.

The term “food-grade bacterium” means a bacterium that is compatible with administration in a food.

The term “probiotic” means preparations of microbial cells or of components of microbial cells which have a beneficial effect on the health or well-being of the host. (Salminen S, Ouwehand A. Benno Y. et al. “Probiotics: how should they be defined” Trends Food Sci. Technol. 1999: 10, 107-10)

The term “prebiotic” denotes food substances that promote the growth of probiotics in the intestine. They are not decomposed in the stomach and/or in the upper intestine or absorbed in the digestive tract of the person who ingests them, but are fermented by the gastrointestinal microflora and/or by the probiotics. Prebiotics are defined, for example, by Glenn R. Gibson and Marcel B. Roberfroid, “Dietary Modulation of the Human Colonic Microbiota: Introducing the Concept of Prebiotics”, J. Nutr. 1995 125: 1401-1412.

Combination of Active Agents

a) Petroselinic Acid

According to a first embodiment variant, petroselinic acid or monounsaturated fatty acid (C18:1 n-12 or cis delta 6) or C18 delta-6-cis-octadecenoic acid, may be used in an isolated form.

According to another variant of the invention, the petroselinic acid is used in the form of a plant extract containing same, such as an oil. This form is particularly suitable for oral administration.

The petroselinic acid-rich oils are more particularly chosen from umbellifera plant oils.

The expression “petroselinic acid-rich oil” means an oil comprising at least 40% of petroselinic acid.

Umbellifera plants are plants whose flowers are arranged in umbels, and the species that are particularly rich in petroselinic acid are Umbellifarea-Apiacea and Araliaceae. Plants of the Thapsia genus are also sources of petroselinic acid (Avato et al., Lipids, 2001, 36, 845).

The species preferably used in the invention are coriander, chervil, carrot, celery, cumin, caraway, parsley and dill, or mixtures thereof. The petroselinic acid-source umbellifera plant oil that is most particularly suitable for use in the invention may be extracted from the seed of these umbellifera plants, for example by milling or pressing, and then refining.

The umbellifera plant oil has a petroselinic acid content which varies according to the umbellifera plant seed from which it is extracted. For the same umbellifera plant, the petroselinic acid content also varies according to the country of origin of the umbellifera plant and according to the extraction, which may be more or less complete.

Petroselinic acid is also an abundant compound (approximately 48%) of Geranium sanguineum seed oil.

In particular, petroselinic acid may be used in the form of an umbellifera plant oil or Geranium sanguineum oil.

Thus, according to one embodiment, the umbellifera plant oil more particularly considered in the invention may be chosen from dill, parsley, caraway, cumin, celery, carrot, chervil and coriander seed oils, and mixtures thereof.

Preferably, petroselinic acid is used in the form of a coriander seed oil. According to the present invention, coriander seed oils are covered by the expression “coriander oil”.

The contents are variable depending on whether the combination of active agents in accordance with the invention is used in a medicament for oral administration or a functional food.

The petroselinic acid content, in the medicament intended for oral administration in accordance with the invention may be between 0.01% and 70% by weight, especially between 0.1% and 70% by weight, and particularly between 1% and 70% by weight, relative to the total weight of the medicament.

The petroselinic acid content, in a functional food in accordance with the invention may be between 0.05% and 2% by weight, especially between 0.1% and 1% by weight, and particularly between 0.3% and 0.6% by weight, relative to the total weight of the functional food.

The petroselinic acid content in a medicament intended for oral administration or in a functional food in accordance with the invention may be such that the daily dose of said petroselinic acid is between 0.5 and 2000 mg/day, particularly between 1 and 1000 mg/day, and especially between 5 and 700 mg/day.

b) Taurine

A combination of active agents according to the invention may comprise taurine, or hypotaurine. It may also use a salt thereof. The salts that may be used are obviously chosen for their total harmlessness. Alkali metal or alkaline-earth metal salts, in particular magnesium salts, manganese, iron(II) or zinc salts are suitable in this respect.

The content of taurine, hypotaurine or a salt thereof in a medicament intended for oral administration in accordance with the invention or in a functional food in accordance with the invention may be such that the daily dose of said taurine, hypotaurine or a salt thereof is between 1 and 700 mg/day, particularly between 10 and 500 mg/day and especially between 50 and 300 mg/day.

c) Zinc

The term “zinc” means zinc or a salt thereof (zinc acetate, chloride, citrate, lactate, gluconate, lactate, oxide, carbonate or sulfate), in particular Zn(II) salts, preferably complexed with one or more (poly)hydroxy acids such as gluconate.

The term “(poly)hydroxy acid” means any carboxylic acid which comprises a hydrocarbon-based chain which is linear or branched, and saturated or unsaturated, preferably saturated and/or linear, comprising from 1 to 10 carbon atoms and from 1 to 9 hydroxyl groups, and comprising from 1 to 4 carboxylic groups —C(O)—OH, at least one of said —C(O)—OH functions of which is in the carboxylate form —C(O)—O— complexed with the Zn atom, preferably Zn(II).

More particularly, the zinc salt is complexed with two carboxylate groups such as that of formula (I):

R—C(O)—O—Zn—O—C(O)—R′ (I)

in which R and R′, which may be identical or different, represent a (C₁-C₆)(poly)hydroxyalkyl group,

and also the solvates thereof, such as hydrates, and the enantiomers thereof,

Preferably, the compound of formula (I) is zinc gluconate.

According to a particular embodiment of the invention, the zinc is not a zinc oxide, but a zinc salt. The term “Zn(II)” means a zinc atom in oxidation state Zn²⁺.

The content of zinc gluconate in a medicament intended for oral administration in accordance with the invention or in a functional food in accordance with the invention may be such that the daily dose of said zinc gluconate is between 0.01 and 300 mg/day, especially between 0.1 and 200 mg/day, and in particular between 1 and 100 mg/day.

d) Lycopene

A combination of active agents according to the invention may also comprise lycopene.

Lycopene is a natural pigment found in ripe fruit, particularly in tomato, but it also exists in synthetic form, especially synthesized from a fungus, Blakeslea trispora.

It belongs to the carotenoid family and its structure is similar to that of β-carotene.

It may in particular be sold by the company Lycored under the name Lyc-O-Mato®.

Preferably, lycopene is used in a combination of active agents in accordance with the invention. In other words, the combination of active agents comprises, or even consists of, petroselinic acid and lycopene.

The lycopene content in a medicament intended for oral administration in accordance with the invention or in a functional food in accordance with the invention may be such that the daily dose of lycopene is between 0.01 and 20 mg/day, particularly between 0.1 and 15 mg/day, and especially between 0.5 and 10 mg/day.

The active agent or the combination of active agents in accordance with the present invention, may also be used with additional active agents suitable for the mode of administration considered, as is described hereinbelow.

In particular, the medicament or the functional food may also comprise vitamin D3 and/or tocopheryl acetate.

Thus, according to a preferred embodiment of the invention, petroselinic acid or the combination of active agents in accordance with the present invention is used in a medicament for oral administration or in a functional food, said medicament or functional food comprising petroselinic acid, taurine, zinc, preferably zinc gluconate, vitamin D3 and tocopheryl acetate.

More particularly, the medicaments intended for oral administration in accordance with the present invention may be in any oral-route galenical form normally used.

According to one embodiment, a medicament intended for oral administration in accordance with the invention comprises:
(i) petroselinic acid in a content of between 1% and 70% by weight, especially between 10% and 70% by weight and particularly between 20% and 70% by weight relative to the total weight of the combination of active agents;
(ii) taurine in a content of between 1% and 50% by weight, especially between 5% and 40% by weight and particularly between 10% and 30% by weight relative to the total weight of the combination of active agents; and/or
(iii) at least one zinc (poly)hydroxy acid, preferably zinc gluconate, in a content of between 0.001% and 40% by weight, especially between 0.01% and 25% by weight and particularly between 0.1% and 20% by weight relative to the total weight of the combination of active agents;
(iv) optionally vitamin D3 in a content of between 0.0001% and 1.0% by weight, especially between 0.0001% and 0.5% by weight and particularly between 0.0001% and 0.1% by weight relative to the total weight of the combination of active agents; and
(v) optionally tocopheryl acetate in a content of between 0.01% and 10% by weight, especially between 0.1% and 10% by weight and particularly between 0.2% and 5% by weight relative to the total weight of the combination of active agents.

According to a particular embodiment, a medicament for oral administration in accordance with the invention comprises ingredients i) to v), taken together or individually:
(i) petroselinic acid in a content of between 1% and 70% by weight, especially between 10% and 70% by weight and particularly between 15% and 70% by weight relative to the total weight of the medicament;
(ii) taurine in a content of between 1% and 40% by weight, especially between 5% and 40% by weight and particularly between 5% and 30% by weight relative to the total weight of the medicament; and/or
(iii) at least one zinc (poly)hydroxy acid, preferably zinc gluconate, in a content of between 0.001% and 30% by weight, especially between 0.01% and 25% by weight and particularly between 0.1% and 20% by weight relative to the total weight of the medicament;
(iv) optionally vitamin D3 in a content of between 0.0001% and 1.0% by weight, especially between 0.0001% and 0.5% by weight and particularly between 0.0001% and 0.1% by weight relative to the total weight of the medicament; and
(v) optionally tocopheryl acetate in a content of between 0.01% and 10% by weight, especially between 0.1% and 10% by weight and particularly between 0.2% and 5% by weight relative to the total weight of the medicament.

According to a particular embodiment, the medicament for oral administration comprises all of the abovementioned ingredients (i) to (iii).

According to a particular embodiment, the medicament for oral administration comprises all of the abovementioned ingredients (i) to (v).

The formulation of the medicament in accordance with the invention may be performed via the usual processes for producing coated tablets, gel capsules, gels, emulsions, tablets, lozenges or wafer capsules.

The medicaments according to the invention, intended for oral administration, may especially comprise all or only a part of the daily dose.

In other words, one to three medicaments may be administered per day.

Typically, the duration of this treatment for oral administration may be greater than 4 weeks, especially from 4 to 15 weeks, with, where appropriate, one or more periods of stoppage which may range from a few days to several months.

Needless to say, a person skilled in the art will take care to select the optional additional additives and/or the amount thereof such that the advantageous properties of the combination according to the invention or of the medicament comprising the combination according to the invention are not, or are not substantially, adversely affected by the envisaged addition.

The medicament or functional food in accordance with the present invention, which has been grouped under the term “composition” in the passage which follows for the sake of simplicity, may also contain protective hydrocolloids (such as gums, proteins or modified starches), binders, film-forming agents, encapsulating agents/materials, wall/shell materials, matrix compounds, coating agents, emulsifiers, surfactants, solubilizers (oils, fats, waxes, lecithins, etc.), adsorbents, “carriers”, fillers, co-compounds, dispersants, wetting agents, processing aids (solvents), flow agents, taste-masking agents, bulking agents, gelling agents, gellants, antioxidants and antimicrobial agents. The composition may also contain conventional pharmaceutical additives and adjuvants, excipients and diluents, including, but not limited to, water, gelatin of any origin, plant gums, lignin sulfonate, talc, sugars, starch, gum arabic, plant oils, polyalkylene glycols, flavorings, preserving agents, stabilizers, emulsifiers, buffers, lubricants, colorants, wetting agents, fillers and the like. In all cases, these other components are chosen as a function of their intended use.

The composition may be a whole nutritional formula.

The composition according to the invention may comprise a source of proteins.

Any suitable food protein may be used, for example proteins of animal origin (such as milk proteins, meat proteins and egg proteins), plant proteins (such as soybean proteins, wheat proteins, rice proteins and pea proteins), mixtures of free amino acids, or combinations thereof. Milk proteins such as casein and lactoserum, and soybean proteins, are particularly preferred.

The proteins may be intact or hydrolyzed or may take the form of a mixture of intact and hydrolyzed proteins. It may be desirable to provide partially hydrolyzed proteins (degree of hydrolysis of between 2% and 20%), for example for animals suspected of being at risk of developing an allergy to cow's milk. If hydrolyzed proteins are required, the hydrolysis process may be performed according to techniques known to those skilled in the art. For example, a whey protein hydrolyzate may be prepared by enzymatic hydrolysis of the whey fraction in one or more steps. If the whey fraction used as starting material is substantially free of lactose, it has been found that the protein undergoes a smaller amount of blockage of its lysines during the hydrolysis process. This makes it possible to go from about 15% by weight of blocked lysines to less than 10% by weight of blocked lysines, relative to the total weight of lysines in the protein, for example about 7% by weight of lysines, which makes it possible to appreciably improve the nutritional quality of the source of protein.

The composition may also contain a source of sugars and a source of fat.

If the composition contains a source of fat, the source of lipids preferably provides from 5% to 40% of the energy of the composition, for example from 20% to 30% of the energy. A suitable fat profile may be obtained by using a mixture of canola oil, corn oil and sunflower oil with a high content of oleic acid.

A source of carbohydrates may be added to the composition.

The source of sugars preferably provides from 40% to 80% of the energy of the composition. Any suitable type of sugar or carbohydrate may be used, for example sucrose, lactose, glucose, fructose, dehydrated glucose syrup, maltodextrins, and mixtures thereof. Dietary fiber may also be added. Dietary fiber passes through the small intestine and is not digested by the enzymes and functions as a natural swelling agent and laxative. Dietary fiber may be soluble or insoluble, and generally a mixture of the two types is preferred. Suitable sources include dietary fiber from soybean, pea, oat, pectins, guar gum, gum arabic, fructo-oligosaccharides, galacto-oligosaccharides, sialyl-lactose and oligosaccharide derivatives from milk of animal origin. A preferred mixture of fibers is a mixture of inulin and of short-chain fructo-oligosaccharides. Preferably, if the fiber is present, the fiber content is between 2 and 40 g/l and preferably between 4 and 10 g/l of the ready-to-consume composition.

The composition may also contain minerals and trace elements such as trace elements and vitamins, in accordance with the recommendations of governmental bodies such as USRDA (United States Recommended Daily Allowances). For example, the composition may contain, as a daily dose, one or more of the following trace elements in the indicated ranges: 300 to 500 mg of calcium, 50 to 100 mg of magnesium, from 150 to 250 mg of phosphorus, 5 to 20 mg of iron, from 1 to 7 mg of zinc, from 0.1 to 0.3 mg of copper, 50 to 200 mg of iodine, 5 to 15 μg of selenium, from 1000 to 3000 μg of beta-carotene, from 10 to 80 mg of vitamin C, from 1 to 2 mg of vitamin B1, 0.5 to 1.5 mg of vitamin B6, 0.5 to 2 mg of vitamin B2, from 5 to 18 mg of niacin, from 0.5 to 2.0 μg of vitamin B12, from 100 to 800 μg of folic acid, 30 to 70 μg of biotin, from 1 to 5 μg of vitamin D, from 3 to 10 μg of vitamin E.

One or more food-grade emulsifiers may be incorporated into the composition. For example, esters of diacetyltartaric acid of mono- and diglycerides, of lecithin and of mono- and diglycerides. Similarly, suitable salts and stabilizers may be included.

The composition may be administered in the form of a powder for reconstitution with milk or water.

Preferably, the composition is in the form of a powder, for example a long-life powder. The long life may be obtained, for example, by providing the composition with a water activity of less than 0.2, for example from about 0.19 to 0.05, preferably less than 0.15.

The water activity or “a_{w}” is a measure of the energy state of water in a system. It is defined as being the vapor pressure of water divided by that of pure water at the same temperature. Consequently, pure distilled water has a water activity of exactly 1.

The composition described above may be prepared according to any suitable process. For example, it may be prepared by mixing together the proteins, the source of carbohydrates and the source of fat in suitable proportions. If they are used, the emulsifiers may be incorporated at this stage. The vitamins and minerals may be added at this stage, but are generally added later to avoid thermal degradation. All the lipophilic vitamins, emulsifiers and analogs may be dissolved in the source of fats before mixing. Water, preferably water that has been subjected to reverse osmosis, may then be mixed so as to form a liquid mixture. The temperature of the water is advantageously between about 50° C. and about 80° C. to facilitate the dispersion of the ingredients. Commercially available liquefying agents may be used to form the liquid mixture. The liquid mixture is then homogenized, for example in two steps.

The liquid mixture may then be heat-treated to reduce the bacterial load, by rapidly heating the liquid mixture to a temperature in the range from about 80° C. to about 150° C. for about 5 seconds to about 5 minutes, for example. This may be performed by steam injection, autoclave or by heat exchanger, for example a plate heat exchanger.

Next, the liquid mixture may be cooled to about 60° C. to about 85° C., for example by instantaneous cooling. The liquid mixture may then be homogenized again, for example in two steps from about 10 MPa to about 30 MPa in the first step and from about 2 MPa to about 10 MPa in the second step. The homogenized mixture may then be cooled so as to be able to add heat-sensitive components, such as the vitamins and minerals. The pH and the solids content of the homogenized mixture are conventionally adjusted at this stage.

The homogenized mixture is transferred to a suitable drying machine, such as a spray dryer or lyophilizer and converted into powder. The powder must have a moisture content of less than about 5% by weight.

In one embodiment of the present invention, the animals treated with the composition in accordance with the present invention are at least 2 years old. This age limit applies in particular to man. If the animals treated with the composition in accordance with the present invention are cats or dogs, for example, they are advantageously at least 4 months old.

When the composition is a medicament, the doses of active agents may be adjusted by a doctor.

When the composition is a functional food. Access to this functional food is available to all. As a result, the treatment of obesity might be started at a much earlier stage.

In addition, the fact that the active agent(s) are used in a functional food makes them more pleasant to consume. Examples of functional foods that may be applied to the present invention are yogurts, milk, flavored milk, ice cream, ready-made desserts, powders for reconstitution with, for example, milk or water, chocolate-flavored dairy drinks, malted drinks, ready-made meals, instant meals or drinks for humans or food compositions representing a whole or partial food in the diet of pets or livestock.

Consequently, in one embodiment, the composition in accordance with the present invention is a food product or functional food intended for man, pets or cattle. In particular, the composition is intended for animals selected from the group consisting of dogs, cats, pigs, cattle, horses, goats, sheep, poultry and humans, and in a preferred embodiment, the food product is intended for humans, in particular adult humans.

The composition of the present invention may also comprise at least food-grade bacteria or yeast. The food-grade bacteria may be probiotic bacteria and are preferably chosen from the group consisting of lactic acid bacteria, bifidobacteria, propionibacteria or mixtures thereof. The probiotic bacteria may be lactic acid bacteria or bifidobacteria with established probiotic characteristics. For example, they may also be capable of promoting the growth of a bifidogenic intestinal flora. Suitable probiotic bifidobacteria strains include Bifidobacterium lactis CNCM 1-3446 sold especially by the Danish company Christian Hansen under the brand name Bb12, Bifidobacterium longum ATCC BAA-999 sold by Morinaga Milk Industry Co. Ltd, Japan, under the brand name BB536, the strain Bifidobacterium breve sold by Danisco under the brand name Bb-03, the strain Bifidobacterium breve sold by Morinaga under the brand name M-16V and the strain Bifidobacterium breve sold by Institut Rosell (Lallemand) under the brand name r0070. A mixture of suitable lactic acid bacteria and of bifidobacteria may be used.

As food-grade yeast, mention may be made of Saccharomyces cerevisiae and/or Saccharomyces boulardii.

According to a particular embodiment of the invention, the composition may also contain at least one prebiotic. The prebiotics may promote the growth of certain food-grade bacteria, in particular probiotic bacteria, when they are present in the composition. Preferably, such prebiotics are chosen from oligosaccharides and optionally contain fructose, galactose, mannose, soybean and/or inulin, dietary fiber, or mixtures thereof.

Other characteristics and advantages of the invention will emerge more clearly from the examples that follow, which are given as non-limiting illustrations.

### EXAMPLE 1

### Oral Composition in Soft Capsule Form

| | (mg/soft |
|---|---|
| Ingredients | capsule) |
| Coriander seed oil (65% | 300.00 |
| petroselinic acid) | |
| Taurine | 76.10 |
| Zinc gluconate | 25.75 |
| Vitamin E | 4.10 |
| Vitamin D3 | 0.115 |
| Excipients | |
| Refined coconut oil | 112.00 |
| Yellow beeswax, Cera | 22.000 |
| flava | |
| Sunflower lecithin | 10.00 |
| Capsule | |
| Fish gelatin | 144.6 |
| Glycerol | 58.6 |
| Purified water | 6.8 |

### EXAMPLE 2

### Oral Composition as a Stick in Emulsion Form

| Ingredients | (g/stick) |
|---|---|
| Coriander seed oil | 0.65 |
| (of which 65% of | |
| petroselinic acid) | |
| Vitamin E | 0.0082 |
| Excipients | |
| Water | 1.722 |
| Sugar | 0.911 |
| Fructose | 0.911 |
| Microcrystalline | 0.032 |
| cellulose | |
| Sodium | 0.004 |
| carboxymethylcellulose | |
| Natural mixture of | 0.034 |
| tocopherols | |
| Sunflower oil | 3.015 |
| Natural lemon flavoring | 0.034 |
| Potassium sorbate | 0.013 |
| Citric acid | 0.013 |
| Propylene glycol | 0.010 |
| alginate | |

### EXAMPLE 3

### Demonstration of the Activating Effect of Petroselinic Acid and of the Combination of Petroselinic Acid with Lycopene According to the Invention on the Synthesis/Basal Release of Lipoxin A4 by Keratinocytes

Mononuclear blood cells are cultured under 5% CO₂ and at 37° C. in a serum-free medium for macrophages (SFM Macrophage; Invitrogen 12065074) for 24 hours.

After this step, the medium is replaced with the same fresh test medium also containing the active agents at the various doses for 30 minutes in the presence of the various products to be evaluated, as indicated in the table of results below. The inflammatory response was then triggered in the presence of phorbol myristate (0.05 μM) and calcium ionophore (1 μM) and of a lipid substrate mixture composed of docosahexaenoic acid (DHA—1 μg/mL) and eicosapentaenoic acid (EPA—1 μg/mL).

The supernatants were then collected after 2 hours of stimulation and frozen at −80° C. before preparation for analysis by mass spectrometry.

Experimental triplicates (three wells) were prepared per experimental condition. Into each culture plate was placed a control corresponding to cells stimulated with the PMA/A23187 mixture and/or with addition of the equimolar mixture of fatty acids.

The thawed supernatants were concentrated by solid-phase extraction (SPE) and taken up in methanol before spectrometric analysis. The analytical method used consists in separating the various analytes by high-pressure liquid chromatography as a function of their retention time and in quantifying them by mass spectrometry.

The analyses were performed using an LC 1290 Infinity chain (Agilent Technologies) coupled to a 6460 Triple Quad LC/MS mass spectrometer (Agilent Technologies) equipped with an electrospray ionization source (Jet stream technology) operating in negative mode. The chromatographic separations were performed on a ZorBAX SB-C18 column.

The results were obtained in pg/mL of cell supernatant. These raw data were then transformed by calculation to obtain the percentage of activation (or of inhibition) of the plate relative to the control using the following calculation:

% modulation=100×(value obtained with the active agent−value of the control)/value of the control

These percentages of modulation are reported in the table of results below.

A combination of active agents in accordance with the invention comprising coriander oil, rich in petroselinic acid, and Lycomato, rich in lycopene, and also these same compounds individually, were tested in accordance with that indicated above.

The results obtained after these comparative tests are as follows:

| | Level of lipoxin A4 |
|---|---|
| Compounds tested | production |
| Coriander oil | +19% |
| (of which between 60% and 75% of | |
| petroselinic acid) | |
| 0.25 mg/ml | |
| Lycomato (containing 10% of | +0% |
| lycopene) | |
| 0.001 mg/ml | |
| Lycomato (containing 10% of | +90% |
| lycopene) | |
| 0.001 mg/ml + coriander oil | |
| (of which between 60% and 75% of | |
| petroselinic acid) 0.25 mg/ml | |

It is observed that coriander oil containing petroselinic acid stimulates the production of lipoxin A4.

Lycomato alone does not, itself, lead to any variation in the level of production of lipoxin A4.

However, it may be seen that the effect of a combination in accordance with the invention on the production of lipoxin A4 is very markedly greater than the sum of the effects of the compounds used individually.

Specifically, an increase in the production of lipoxin A4 of 90% relative to the basal level of production of this anti-inflammatory component could be seen when the lymphocyte cells tested were placed in contact with the combination of active agents.

It is thus indeed a synergistic effect of a combination of active agents in accordance with the invention that is observed and demonstrated here.

### EXAMPLE 4

### Demonstration of the Activating Effect of Petroselinic Acid and of the Combination of Petroselinic Acid with Taurine According to the Invention on the Synthesis/Basal Release of Lipoxin A4 by Keratinocytes

A protocol similar to that described in Example 3 was performed, replacing, however, the Lycomato with taurine.

Thus, a combination of active agents in accordance with the invention comprising coriander oil, rich in petroselinic acid, and taurine, and also these same compounds individually, were tested in accordance with that indicated below.

The results obtained after these comparative tests are as follows:

| | Level of lipoxin A4 |
|---|---|
| Compounds tested | production |
| Coriander oil | +19% |
| (of which between 60% and 75% | |
| of petroselinic acid) | |
| 0.25 mg/ml | |
| Taurine | +38% |
| 3.1 mg/ml | |
| Taurine 3.1 mg/ml + coriander | +118% |
| oil | |
| (of which between 60% and 75% | |
| of petroselinic acid) 0.25 mg/ml | |

It is thus observed that coriander oil containing petroselinic acid stimulates the production of lipoxin A4.

In this case also, it may be seen that the effect of a combination in accordance with the invention on the production of lipoxin A4 is very markedly greater than the sum of the effects of the compounds used individually.

Specifically, an increase in the production of lipoxin A4 of 94% relative to the basal level of production of this anti-inflammatory component could be seen when the lymphocyte cells tested were placed in contact with the combination of active agents.

It is thus indeed a synergistic effect of a combination of active agents in accordance with the invention that is observed and demonstrated here.

### EXAMPLE 5

The results obtained after these comparative tests are as follows:

A protocol similar to that described in Example 3 was performed, replacing, however, the Lycomato with zinc gluconate.

Thus, a combination of active agents in accordance with the invention comprising coriander oil, rich in petroselinic acid, and zinc gluconate, and also these same compounds individually, were tested in accordance with that indicated below.

| | Level of lipoxin A4 |
|---|---|
| Compounds tested | production |
| Coriander oil | +19% |
| (of which between 60% and | |
| 75% of petroselinic acid) | |
| 0.25 mg/ml | |
| Zinc gluconate | −5% |
| 0.005 mg/ml | |
| Zinc gluconate 0.005 mg/ml + | +94% |
| Coriander oil | |
| (of which between 60% and | |
| 75% of petroselinic acid) | |
| 0.25 mg/ml | |

In this case also, it may be seen that the effect of a combination in accordance with the invention on the production of lipoxin A4 is very markedly greater than the sum of the effects of the compounds used individually.

Specifically, an increase in the production of lipoxin A4 of 94% relative to the basal level of production of this anti-inflammatory component could be seen when the lymphocyte cells tested were placed in contact with the combination of active agents.

It is thus indeed a synergistic effect of a combination of active agents in accordance with the invention that is observed and demonstrated here.

### EXAMPLE 6

### Example of Medicament Formulation

| | % by weight relative to the total |
|---|---|
| Components | weight of the composition |
| Petroselinic acid | 54.9 (provided by the coriander seed oil) |
| Zinc gluconate | 6.3 (of which 13.6% of active material) |
| Taurine | 18.7 (of which 98.5% of active material) |
| Vitamin E | 1.0 (of which 67% of active material) |
| Vitamin D3 | 0.03 (of which 2.5% of active material) |

### EXAMPLE 7

### Example of Functional Food Formulation

Chocolate-Flavored Dessert Cream

| | % by weight relative to the |
|---|---|
| | total weight of the |
| Components | composition |
| Fresh powdered skimmed milk | 87.3 |
| Cream 34% fat | 2.1 |
| Sugar | 3.00 |
| Starch | 1.7 |
| Inulin powder | 2.8 |
| Carrageenan | 0.6 |
| Cocoa | 2.0 |
| Flavorings | 0.2 |
| Coriander oil | 0.3 |
| (65% petroselinic acid) | |
| microencapsulated | |

The dairy products (milk, milk powder and cream) are placed in a container maintained at low temperature (8-15° C.) and mixed to hydrate the dairy ingredients. The mixture is then heated to a temperature ranging up to 68-75° C. and the other ingredients: sugar, starch, inulin, carrageenan, are incorporated.

The whole is then mixed for about 30 minutes until the various ingredients have fully dissolved.

A homogenization step using an APV Gaulin GmbH Homogenisator (type: 132MC4 5TBSX) is performed by applying a pressure of 50-120 bar by using a feed pressure of 4 bar. The product is then sterilized at 130° C. for 20-30 seconds. The product is then cooled to 5-10° C. and then dosed.

Recommended dose: 2 pots of dessert cream per day

### EXAMPLE 8

### Example of Formulation of a Drink Forming a Functional Food

Tea-Flavored Drink

| | % by weight relative to the |
|---|---|
| | total weight of the |
| Components | composition |
| Tea powder | 0.110% |
| Citric acid | 0.190% |
| Preserving agents | 0.063% |
| Sweeteners | 0.039% |
| Lemon flavoring | 0.010% |
| Tea flavoring | 0.010% |
| Coriander oil | 0.03% |
| (65% petroselinic acid) | |
| microencapsulated | |
| Water | 99.548% |

Recommended dose: 1 liter per day

1-13. (canceled)

14. Method for promoting weight loss and/or weight maintenance in man and/or animals in need thereof, comprising an oral administration step of petroselinic acid or a combination of active agents comprising at least petroselinic acid and at least one compound chosen from zinc, taurine, a salt thereof, lycopene, and mixtures thereof, wherein petroselinic acid or said combination is a medicament or a functional food.

15. The method as claimed in claim 14, wherein the medicament or the functional food is intended for reducing the weight of fat mass of a human and/or an animal.

16. The method as claimed in claim 14, wherein the medicament or the functional food is intended for reducing the total weight of a human and/or an animal.

17. The Method as claimed in claim 14, wherein the petroselinic acid is combined with at least taurine or zinc gluconate.

18. The Method as claimed in claim 14, wherein the petroselinic acid is combined with at least taurine and zinc gluconate.

19. Method for combating obesity, comprising an oral administration step of petroselinic acid or a combination of active agents comprising at least petroselinic acid and at least one compound chosen from zinc, taurine, a salt thereof, lycopene, and mixtures thereof, wherein petroselinic acid or said combination is a medicament or a functional food.

20. The method of claim 19, wherein the petroselinic acid is combined with at least taurine or zinc gluconate.

21. The method of claim 19, wherein the petroselinic acid is combined with at least taurine and zinc gluconate.

22. The method as claimed in claim 14, wherein said petroselinic acid is used in an isolated form or in the form of a plant extract containing same.

23. The method as claimed in claim 14, in which said petroselinic acid is used in the form of umbellifera plant oil or Geranium sanguineum oil.

24. The method as claimed in claim 14, in which said umbellifera plant oil is chosen from dill, parsley, caraway, cumin, celery, carrot, chervil and coriander seed oils, and mixtures thereof, preferably in the form of a coriander seed oil.

25. The method as claimed in claim 14, in which said umbellifera plant oil is in the form of a coriander seed oil.

26. The method as claimed in claim 14, in which the combination of active agents is used in a medicament for oral administration or in a functional food, said medicament or functional food also comprising vitamin D3 and/or tocopheryl acetate.

27. The method as claimed in claim 14, in which the combination of active agents is used in a medicament for oral administration or in a functional food, said medicament or functional food also comprising a food-grade bacterium and/or a yeast.

28. The method as claimed in claim 14, wherein the food-grade bacterium is chosen from lactic acid bacteria, bifidobacteria and propionibacteria, and mixtures thereof.

29. The method as claimed in claim 14, wherein said petroselinic acid or said combination is used in a medicament for oral administration comprising petroselinic acid in a content of between 0.01% and 70% by weight, relative to the total weight of the medicament.

30. The method as claimed in claim 14, wherein said petroselinic acid or said combination is used in a medicament for oral administration comprising petroselinic acid in a content of between 1% and 70% by weight, relative to the total weight of the medicament.

31. The method as claimed in claim 14, wherein said petroselinic acid or said combination is used in a functional food comprising petroselinic acid in a content of between 0.05% and 2% by weight, relative to the total weight of the functional food.

32. The method as claimed in claim 14, wherein said petroselinic acid or said combination is used in a functional food comprising petroselinic acid in a content of between 0.3% and 0.6% by weight, relative to the total weight of the functional food.
